(19) United States
(12) Patent Application Publication
(10) Pub. No.: US 2022/0260597 A1
(43) Pub. Date: Aug. 18, 2022

(54) METHODS FOR THE TREATMENT OF FAMILIAL HETEROZYGOUS AND HOMOZYGOUS HYPERCHOLESTEROLEMIA WITH CYCLODEXTRINS

(71) Applicant: BEREN THERAPEUTICS P.B.C., West Hollywood, CA (US)

(72) Inventors: Diana KERWIN, Dallas, TX (US); Jason CAMM, Los Angeles, CA (US); Henricus DUCKERS, San Francisco, CA (US)

(21) Appl. No.: 17/675,170

(22) Filed: Feb. 18, 2022

Related U.S. Application Data
(60) Provisional application No. 63/150,908, filed on Feb. 18, 2021.

(51) Int. Cl. G01N 33/92; A61P 3/06

(52) U.S. Cl. CPC G01N 33/92; A61P 3/06

(57) ABSTRACT

Disclosed herein are methods alleviating or reducing inflammation and/or oxidative stress induced by oxidized LDL, reducing an amount (e.g., concentration) total cholesterol, reducing accumulation of total LDL, reducing an amount (e.g., concentration) of and/or a size (e.g., average size, maximum size) of, and/or changing the shape of circulating (e.g., blood, serum, plasma) cholesterol crystals (and/or clots comprising cholesterol crystals) in an individual. Further disclosed herein are methods of treating familial hypercholesterolemia, reducing statin, cholesterol uptake inhibitor or PCSK9 inhibitor treatment, or reducing a frequency of, or delaying plasmapheresis treatment of a subject diagnosed with or suspected to have familial hypercholesterolemia. The methods generally involve administering a therapeutically effective amount of 2-hydroxypropyl-beta-cyclodextrin to the individual. Further provided herein are pharmaceutical compositions comprising a therapeutically effective amount of 2-hydroxypropyl-beta-cyclodextrin and a pharmaceutically acceptable excipient.

CROSS-REFERENCE

This application claims the benefit of U.S. Provisional Application No. 63/150,908 filed Feb. 18, 2021, which is incorporated herein by reference in its entirety.

### BACKGROUND

Familial hypercholesterolemia (FH) is a genetic disorder that belongs to the dyslipidemic diseases. FH can be segregated into the rare homozygous (HoFH; Prevalence 1:1,000,000) and the more frequent, heterozygous FH (HeFH; prevalence: 1:200-1:500). Heterozygous FH (HeFH) patients have an estimated prevalence approximately 1 in 500 individuals in Europe. In the United States, prevalence is 1:311 individuals, or over one million individuals. It should be noted that some of these estimates likely include individuals with polygenic hypercholesterolemia (patients without true FH) and that HeFH often goes unrecognized. FH increases the chance of atherosclerotic cardiovascular disease at an early age, therefore, strongly reducing life expectancy.

### SUMMARY

There is a need for effective treatments of heterozygous as well as homozygous familial hypercholesterolemia and/or symptoms thereof. This disclosure addresses this unmet medical need.

In one aspect, the present disclosure provides a method of i) alleviating or reducing inflammation and/or oxidative stress induced by oxidized LDL, ii) reducing an amount (e.g., concentration) total cholesterol, iii) reducing accumulation of total LDL, iv) reducing an amount (e.g., concentration) of and/or a size (e.g., average size, maximum size) of, and/or changing the shape of circulating (e.g., blood, serum, plasma) cholesterol crystals (and/or clots comprising cholesterol crystals), or promoting renal (and/or) hepatogenic clearance of cholesterol (derivates) in an individual. The method comprises administering a therapeutically effective amount of cyclodextrin or a derivative thereof to the individual, thereby alleviating or reducing inflammation and/or oxidative stress induced by oxidized LDL, and/or reducing the amount of and/or size of, and/or changing the shape of circulating cholesterol crystals (and/or clots comprising cholesterol crystals) in the individual diagnosed with or suspected to have familial hypercholesterolemia, and/or promoting renal (and/or) hepatogenic clearance of cholesterol (derivates) in an individual. In some cases, the size (e.g., average size, maximum size) of circulating (e.g., blood, serum, plasma) cholesterol crystals (and/or clots comprising cholesterol crystals) is reduced by at least about 10% (e.g., at least about 15%, at least about 20%, at least about 25%, at least about 30%, at least about 35%, at least about 40%, at least about 45%, at least about 50%, or greater) relative to the size (e.g., average size, maximum size) of circulating (e.g., blood, serum, plasma) cholesterol crystals (and/or clots comprising cholesterol crystals) prior to treatment with the cyclodextrin or the derivative thereof. In some cases, the cyclodextrin or derivative thereof comprises 2-hydroxypropyl-beta-cyclodextrin. In some cases, the amount (e.g., concentration) of circulating (e.g., blood, serum, plasma) cholesterol crystals (and/or clots comprising cholesterol crystals) is reduced by at least about 10% (e.g., at least about 15%, at least about 20%, at least about 25%, at least about 30%, at least about 35%, at least about 40%, at least about 45%, at least about 50%, or greater) relative to the amount (e.g., concentration) of circulating (e.g., blood, serum, plasma) cholesterol crystals (and/or clots comprising cholesterol crystals) prior to treatment with the cyclodextrin or the derivative thereof. In some cases, the treatment reduces severity or a symptom of familial hypercholesterolemia in the individual or reduces the clinical manifestation of cardiovascular disease or incidence of major cardio-and cerebrovascular complications including, but not limited to, stroke, transient ischemic attack (TIA), angina, myocardial infarction, ischemic heart failure, claudication or gangrene. In some cases, the treatment increases renal and/or hepatogenic clearance of cholesterol or cholesterol derivates in the individual by at least about 10% (e.g., at least about 15%, at least about 20%, at least about 25%, at least about 30%, at least about 35%, at least about 40%, at least about 45%, at least about 50%, at least about 75%, at least about 100%, at least about 200%, at least about 300%, at least about 400%, at least about 500%, or greater) relative to the amount of cholesterol and/or cholesterol derivates cleared prior to the treatment.

The present disclosure also provides a method of treating familial hypercholesterolemia and/or one or more symptoms thereof in an individual, or treating an individual suspected to have familial hypercholesterolemia and/or one or more symptoms thereof. The method comprises administering a therapeutically effective amount of cyclodextrin, or derivative thereof (e.g., 2-hydroxypropyl-beta-cyclodextrin), to the individual, thereby treating the familial hypercholesterolemia and/or one or more symptoms thereof in the individual. In some cases, the treating comprises reducing a size and/or an amount of circulating (e.g., blood, plasma, serum) cholesterol crystals (and/or clots comprising cholesterol crystals) in the subject. In some cases, the size (e.g., average size, maximum size) of circulating (e.g., blood, serum, plasma) cholesterol crystals (and/or clots comprising cholesterol crystals) is reduced by at least about 10% (e.g., at least about 15%, at least about 20%, at least about 25%, at least about 30%, at least about 35%, at least about 40%, at least about 45%, at least about 50%, or greater) relative to the size (e.g., average size, maximum size) of circulating (e.g., blood, serum, plasma) cholesterol crystals (and/or clots comprising cholesterol crystals) prior to treatment with the 2-hydroxypropyl-beta-cyclodextrin. In some cases, the amount (e.g., concentration) of circulating (e.g., blood, serum, plasma) cholesterol crystals (and/or clots comprising cholesterol crystals) is reduced by at least about 10% (e.g., at least about 15%, at least about 20%, at least about 25%, at least about 30%, at least about 35%, at least about 40%, at least about 45%, at least about 50%, or greater) relative to the amount (e.g., concentration) of circulating (e.g., blood, serum, plasma) cholesterol crystals (and/or clots comprising cholesterol crystals) prior to treatment with the 2-hydroxypropyl-beta-cyclodextrin. In some cases, the treating results in a change in shape of circulating cholesterol crystals (and/or clots comprising cholesterol crystals). In some cases, the treating results in a decrease in inflammation (e.g., as measured by, e.g., cytokine protein and/or RNA levels) as compared to a level of inflammation prior to treatment with the 2-hydroxypropyl-beta-cyclodextrin. In some cases, the treating results in an improvement in dermatologic manifestations as compared to prior to treatment. In some cases, the treatment reduces severity or a symptom of familial hypercholesterolemia in the individual or reduces the clinical manifestation of cardiovascular disease or incidence of major cardio-and cerebrovascular complications including, but not limited to, stroke, TIA, angina, myocardial infarction, ischemic heart failure, claudication or gangrene. In some cases, the treatment increases renal and/or hepatogenic clearance of cholesterol or cholesterol derivates in the individual by at least about 10% (e.g., at least about 15%, at least about 20%, at least about 25%, at least about 30%, at least about 35%, at least about 40%, at least about 45%, at least about 50%, at least about 75%, at least about 100%, at least about 200%, at least about 300%, at least about 400%, at least about 500%, or greater) relative to the amount of cholesterol and/or cholesterol derivates cleared prior to the treatment.

In any one of the preceding aspects, therapeutically effective amount is from about 50 mg/kg to about 8,000 mg/kg. In any one of the preceding aspects, the therapeutically effective amount is from about 4 g to about 250 g. In any one of the preceding aspects, the therapeutically effective amount is an amount sufficient to achieve a serum, plasma, and/or whole blood concentration of 2-hydroxypropyl-beta-cyclodextrin of about 0.01 mM to about 5 mM. In any one of the preceding aspects, the 2-hydroxypropyl-beta-cyclodextrin is selected from the group consisting of: Kleptose® HP Parenteral Grade, Kleptose® HPB Parenteral Grade, Kleptose® HPB-LB Parenteral Grade, Cavitron® W7 HP5 Pharma cyclodextrin, Cavitron® W7 HP7 Pharma cyclodextrin, Trappsol® Cyclo™, and VTS-270/adrabetadex. In any one of the preceding aspects, the subject has one or more risk factors for familial hypercholesterolemia. In some cases, the one or more risk factors for FH is selected from the group consisting of family history of familial hypercholesterolemia, high level of LDL cholesterol in at least one of parents, a change in LDLR gene, ApoB gene, ApoE, SATP1, LDLRAP1/ARH or PCSK9 gene. In any one of the preceding aspects, the subject has one or more analytical lab results associated with familial hypercholesterolemia. In some cases, the one or more analytical lab results associated with familial hypercholesterolemia is increased serum/plasma total cholesterol and/or increased low density lipoprotein (LDL).

In any one of the preceding aspects, the individual is under 1, under 3, under 5 years old, or at least 5 (e.g., at least 10, at least 15, at least 20, at least 25, at least 30, at least 40) years old. In any one of the preceding aspects, the individual is a human. In any one of the preceding aspects, the administering further comprises: (i) administering, at a first time point, a therapeutically effective first dose of 2-hydroxypropyl-beta-cyclodextrin to the individual; and (ii) administering, at a second time point, a therapeutically effective second dose of 2-hydroxypropyl-beta-cyclodextrin to the individual. In any one of the preceding aspects, the second time point is at least 4 hours, at least 6 hours, at least 8 hours, at least 12 hours, at least 1 day, at least 2 days, at least 3 three days, at least 4 days, at least 5 days, at least 6 days, at least 1 week, at least 2 weeks, at least 3 weeks, or at least 4 weeks after the first time point. In any one of the preceding aspects, the administering further comprises administering every 3 days, every 7 days, every 10 days, every 14 days, every 21 days, every 28 days, every 2 months, every 3 months, every 6 months, every 12 months. In any one of the preceding aspects, the administering is by parenteral methods including intravenous, intravascular, intramuscular, subcutaneous, intrathecal, depot, peristaltic pump administration and/or in conjunction to plasmapheresis.

The present disclosure also provides a method of reducing a complication related to familial hypercholesterolemia comprising administering a therapeutically effective amount of cyclodextrin or derivative thereof to the individual, thereby treating the familial hypercholesterolemia and/or one or more symptoms thereof in the individual. In some instances, the complication comprises increased risk on progressive (accelerated or early onset) atherosclerotic disease including coronary artery (acute myocardial infarction (AMI), angina pectoris, ischemic heart failure), peripheral artery (claudication, gangrene, limb amputation), ischemic cerebrovascular disease (transient ischemic attack (TIA), cerebrovascular accidents (CVA)), renal failure, or high blood pressure (hypertension). In some instances, the treating comprises reducing a size (e.g., average size, maximum size) of circulating (e.g., blood, plasma, serum) cholesterol crystals (and/or clots comprising cholesterol crystals) in the subject, reducing an amount (e.g., concentration) of circulating (e.g., blood, plasma, serum) cholesterol crystals (and/or clots comprising cholesterol crystals) in the subject, changing a shape of circulating (e.g., blood, plasma, serum) cholesterol crystals (and/or clots comprising cholesterol crystals) in the subject, reducing inflammation in the subject (e.g., as measured by, e.g., cytokine protein and/or RNA levels), promoting renal or hepatic clearance of cholesterol from circulation, improving dermatologic manifestations in the subject, and/or reducing incidence, severity or a symptom of stroke, TIA, angina, myocardial infarction, ischemic heart failure, claudication or gangrene.

In some aspects, the cyclodextrin or derivative thereof comprises 2-hydroxypropyl-beta-cyclodextrin. In some instances, the 2-hydroxypropyl-beta-cyclodextrin is selected from the group consisting of: Kleptose® HP Parenteral Grade, Kleptose® HPB Parenteral Grade, Kleptose® HPB-LB Parenteral Grade, Cavitron® W7 HP5 Pharma cyclodextrin, Cavitron® W7 HP7 Pharma cyclodextrin, Trappsol® Cyclo™, and/or VTS-270/adrabetadex. In some instances, cyclodextrin or derivative thereof comprises a complexed 2-hydroxypropyl-beta-cyclodextrin, and/or a cyclodextrin polymer. In some aspects, the therapeutically effective amount is from about 50 mg/kg to about 8,000 mg/kg. Alternatively and/or additionally, the therapeutically effective amount is from about 4 g to about 250 g. Alternatively and/or additionally, the therapeutically effective amount is an amount sufficient to achieve a serum, plasma, and/or whole blood concentration of 2-hydroxypropyl-beta-cyclodextrin of about 0.01 mM to about 5 mM.

In some instances, the administering further comprises: (i) administering, at a first time point, a therapeutically effective first dose of 2-hydroxypropyl-beta-cyclodextrin to the individual; and (ii) administering, at a second time point, a therapeutically effective second dose of 2-hydroxypropyl-beta-cyclodextrin to the individual. In some aspects, the administering further comprises administering every 3 days, every 7 days, every 10 days, every 14 days, every 21 days, every 28 days, every 2 months, every 3 months, every 6 months, every 12 months. In some aspects, the administering is by parenteral methods including intravenous, intravascular, intramuscular, subcutaneous, intrathecal, depot, peristaltic pump administration and/or in conjunction to plasmapheresis.

The present disclosure also provides a method of i) reducing statins, cholesterol uptake inhibitors, or PCSK9 inhibitor treatment, or ii) reducing a frequency of or delaying plasmapheresis treatment to a subject diagnosed with or suspected to have familial hypercholesterolemia, comprising: administering a therapeutically effective amount of cyclodextrin or derivative thereof to the subject, thereby treating the familial hypercholesterolemia and/or one or more symptoms thereof in the subject. In some aspects, the cyclodextrin or derivative thereof comprises 2-hydroxypropyl-beta-cyclodextrin. In some instances, the 2-hydroxypropyl-beta-cyclodextrin is selected from the group consisting of: Kleptose® HP Parenteral Grade, Kleptose® HPB Parenteral Grade, Kleptose® HPB-LB Parenteral Grade, Cavitron® W7 HP5 Pharma cyclodextrin, Cavitron® W7 HP7 Pharma cyclodextrin, Trappsol® Cyclo™, and/or VTS-270/adrabetadex. In some aspects, the therapeutically effective amount is from about 50 mg/kg to about 8,000 mg/kg. Alternatively and/or additionally, the therapeutically effective amount is from about 4 g to about 250 g. Alternatively and/or additionally, the therapeutically effective amount is an amount sufficient to achieve a serum, plasma, and/or whole blood concentration of 2-hydroxypropyl-beta-cyclodextrin of about 0.01 mM to about 5 mM.

In some instances, the administering further comprises: (i) administering, at a first time point, a therapeutically effective first dose of 2-hydroxypropyl-beta-cyclodextrin to the individual; and (ii) administering, at a second time point, a therapeutically effective second dose of 2-hydroxypropyl-beta-cyclodextrin to the individual. In some aspects, the administering further comprises administering every 3 days, every 7 days, every 10 days, every 14 days, every 21 days, every 28 days, every 2 months, every 3 months, every 6 months, every 12 months. In some aspects, the administering is by intravenous administration and/or in conjunction to plasmapheresis.

In some instances, the statin, cholesterol uptake inhibitors, or PCSK9 inhibitor treatment is reduced at least 30% compared to before administering the cyclodextrin or derivative thereof. Alternatively and/or additionally, in some instances, the frequency of the plasmapheresis treatment is reduced at least 30% compared to before administering the cyclodextrin or derivative thereof. Alternatively and/or additionally, in some instances, the plasmapheresis treatment is delayed at least 6 months.

In another aspect, a pharmaceutical composition is provided comprising: an amount of cyclodextrin or its derivative thereof (e.g., 2-hydroxypropyl-beta-cyclodextrin) effective alleviate or reduce inflammation and/or oxidative stress induced by oxidized LDL, promote renal and hepatogenic clearance of cholesterol and/or to reduce an amount of and/or a size of, and/or change a shape of circulating (e.g., blood, serum, plasma) cholesterol crystals (and/or clots comprising cholesterol crystals) in an individual diagnosed with or suspected to have familial hypercholesterolemia; and a pharmaceutically acceptable excipient.

In another aspect, a pharmaceutical composition is provided comprising: an amount of cyclodextrin or its derivative thereof (e.g., 2-hydroxypropyl-beta-cyclodextrin) effective treat familial hypercholesterolemia and/or a symptom thereof, in an individual; and a pharmaceutically acceptable excipient.

In any one of the preceding aspects, the pharmaceutical composition is formulated for single dose or repeated administration. In any one of the preceding aspects, the pharmaceutical composition is formulated for parenteral methods of administration including intravenous, intravascular, intramuscular, subcutaneous, intrathecal, depot, peristaltic pump administration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings below.

FIG. 1A shows cholesterol crystal dissolution capacity of 2-hydroxypropyl-beta-cyclodextrin (HPBCD) in plasma sample of HoFH patients.

FIG. 1B shows mRNA expression level of ABCA1 in human peripheral whole blood sample obtained from HoFH patients.

FIG. 1C shows mRNA expression level of SREBP2 in human peripheral whole blood sample obtained from HoFH patients.

FIGS. 2A and 2B show experimental design to evaluate the effect of 2-hydroxypropyl-beta-cyclodextrin (HPBCD) on liver weight in mouse model.

FIG. 3 shows that 2-hydroxypropyl-beta-cyclodextrin (HPBCD) inhibits increase of liver weight in HPBCD-treated mice as compared to untreated mice.

### DETAILED DESCRIPTION OF THE DISCLOSURE

Familial hypercholesterolemia is mostly (80-90%) caused by a genetic defect in chromosome 19, which encodes for the LDL (low density lipoprotein) receptor that is responsible to bind and clean up LDL from the blood to lower or maintain LDL level. Other causes are manifested by mutations in the gene encoding for major structure proteins of LDL, apolipoprotein B (ApoB), ARH/LDLRAP1, ApoE, STAP1 or within Pro-protein-Convertase Subtilisin/Kexin Type 9 (PCSK9), an enzyme responsible for the degradation of LDL receptor. Thus, major symptoms of familial hypercholesterolemia include high levels of LDL cholesterol (LDL-C), early onset atherosclerosis involving the coronary, cerebrovascular and peripheral vasculature, fatty skin deposit (xanthomas) over the hands, elbows, knees, ankles, and/or around the cornea of the eye, and cholesterol deposits in the eyelids (xanthelasma).

Clinical presentation of HoFH include skin and tendon xanthomas, total cholesterol levels typically between 500 and 1,000 mg/dL, and childhood onset of symptomatic coronary disease, as well as aortic valve and proximal aortic root disease. In HoFH, skin and tendon xanthomas are seen by the age of 10, while in HeFH they can appear in young adulthood. Tendon xanthomas are present in more than 70% of patients with FH by age 40 to 50. It is important to note these xanthomas can easily be missed unless specifically sought. The result is early endothelial dysfunction with accelerated atherosclerosis as compared to the other dyslipidemias and, if untreated, to premature cardiovascular disease and death. In the absence of aggressive lipid lowering therapy (e.g., statins with or without other lipid lowering therapies), life span is significantly shortened (e.g., premature death), as the disease already manifests in younger patients with signs of atherosclerotic disease, such as peripheral artery disease (PAD), coronary artery disease (CAD; myocardial infarction, angina pectoris and related post-infarct congestive heart failure) and cerebrovascular artery disease (stroke, TIA). Clinical presentation of heterozygous FH includes patients present with symptoms or signs of cardiovascular disease or adverse cardiovascular disease events at the age of 30-50 years. Many will be identified by an LDL typically greater than the 90th percentile for age and sex when the test was performed for cardiovascular risk screening. The duration and degree of LDL elevation is primarily responsible for the development of atherosclerotic cardiovascular disease (CVD) rather than the specific genetic defect(s) present.

Current guidelines recommend statins in combination with cholesterol uptake inhibitors, as first-line therapy. In children with heterozygous FH, statin therapy does not reduce LDL-C levels to the same degree as adults (about 25 to 40% reduction from baseline depending on dose and potency) but initiation in childhood is imperative to maximizing patient outcomes. However, most FH patients will not achieve goal LDL levels with monotherapy and require multimodal therapies (combinations of statin, ezetimibe, bempedoic acid and/or PCSK9 inhibitors) that are expensive, not currently covered by third-party payors, and results in sub-optimal therapies for most FH patients. For individuals that do not respond to pharmacologic lipid-lowering therapy, LDL apheresis is the only option.

Due to the defects in lipid transport, which is based on impaired (e.g., mutated ApoB) or insufficient (e.g., mutated LDL receptor) lipid transport, the overabundance of LDL and cholesterol accumulates, and attracts phagocytic immune cells such as macrophages, which take up the lipids and turn into foam cells. Foam cells are a hall mark of atherosclerotic disease (plaque foam cells) but also clinically manifest by local accumulations of lipid-laden foamy macrophage, i.e., in xanthomas. It has been widely accepted that LDL is one major risk factor; it carries most of the cholesterol and can turn into highly inflammatory oxidation products (oxLDL, acLDL). Cholesterol-rich LDL particles are taken up by macrophages and comprise the major source for tilting macrophage lipid homeostasis and to turn them into foam cells. Besides overloading the macrophages' lipid storage pool capacity, once ingested LDL-cholesterol precipitates as crystalline cholesterol, which is known to induce local inflammation in the vascular wall.

High amounts of circulating LDL-cholesterol, resulting in inflammatory macrophage/foam cell accumulation/formation through scavenger receptor uptake, intracellular CC deposition can be treated with the administration of HPBCD. Without being limited to any particular mechanism of action, preclinical data suggest that 2-hydroxypropyl-beta-cyclodextrins (HPBCD) could have profound beneficial effects on the pathomechanisms responsible for familial hypercholesterolemia by i) reducing the amount of, and/or size of, and/or changing the shape of cholesterol crystals (and/or clots comprising cholesterol crystals; which can be a potent pro-inflammatory stimulus, ii) increase cholesterol metabolism (oxysterols), iii) improving the reverse cholesterol transport (RCT; via ABC transporters), iv) transcription of an the local anti-inflammatory immune program (by oxysterols production and LXR gene transcriptional activation), v) modulating cholesterol efflux and/or homeostasis and vi) by producing an antioxidative immune environment (by scavenging lipid oxidation) or vii) promoting renal and/or hepatogenic clearance of cholesterol and its derivates. Therefore, 2-hydroxypropyl-beta-cyclodextrins (HPBCD) may provide a novel treatment option for familial hypercholesterolemia and symptoms thereof

Disclosed herein are methods for alleviating or reducing inflammation and/or oxidative stress induced by oxidized LDL, reducing an amount (e.g., concentration) total cholesterol, reducing accumulation of total LDL, and/or improving the renal and/or hepatogenic clearance of cholesterol in an individual, and/or reducing the amount of and/or the size of, and/or changing the shape of circulating (e.g., blood, plasma, serum) cholesterol crystals (and/or clots comprising cholesterol crystals) in an individual (e.g., a human). In some cases, the methods involve treating familial hypercholesterolemia (e.g., by preventing the development and progression of atherosclerotic artery disease, including increase in atherosclerotic plaque lesion load, plaque destabilization, and potential plaque rupture leading to acute vascular occlusion). In some cases, the methods involve treating symptom and/or a clinical manifestation of familial hypercholesterolemia. In some cases, the methods involve treating ischemia to various organs and/or tissues caused by, e.g., familial hypercholesterolemia. In some cases, the methods involve converting inflamed atherosclerotic plaques with a lipid core (vulnerable plaque, high cholesterol plaque) with a high incidence of plaque rupture, to a more fibrotic atherosclerotic plaque (low inflammation, low necrotic core) with a low incidence of plaque rupture (and cardiovascular complications). In some cases, the methods involve improving quality of LDL particles and/or reducing/shielding the inflammatory properties of highly oxidized LDL. Generally, the methods provided herein involve administering a therapeutically effective amount of a cyclodextrin or derivative thereof to a subject in need thereof (e.g., a subject having elevated levels of circulating cholesterol crystals (and/or clots comprising cholesterol crystals)). In some aspects, a cyclodextrin derivative as used herein refers a chemically modified cyclodextrin molecule. For example, a cyclodextrin derivative includes a cyclodextrin molecule in which at least one -OH group is substituted with a hydroxypropyl group. In a particular aspect, the cyclodextrin derivative is 2-hydroxypropyl-beta-cyclodextrin. In another aspect of the invention, the cyclodextrin derivative may be a mixture of two or more 2-hydroxypropyl-beta-cycloxtrin molecule fractions with varying degrees of substitution (with for example, hydroxypropyl groups).

In one aspect, cyclodextrin or derivative thereof is able to reduce cholesterol crystals formation and promote CC resorption and dissolution, improve LDL cholesterol transport into the circulation, increase endogenous cholesterol metabolisms towards oxysterols (e.g., 24,-25-, 27-hydroxycholesterol; 24S-, 25-, 27-OHC) and the local; activation of LXR transcription factor-regulated genes, including genes encoding for the reverse cholesterol transporters ABCA1 and ABCG1, which will result in improved reverse cholesterol transport (RCT) and, improved cholesterol clearance, and thus, reduced atherosclerotic plaque burden and overall risk of atherosclerotic disease and its consequences. Consequentially, a reduction in clinical manifestations of FH through reduction of cardiovascular disease and its consequences, including reduction in skin, tendon, lipid depositions (xanthomas), but also the reduction of clinical signs and symptoms of peripheral artery disease (claudication, gangrene), cerebrovascular disease (Stroke, TIA) and cardiovascular disease (angina, myocardial infarction and congestive heart failure) in an individual diagnosed with or suspected to have familial hypercholesterolemia is provided.

### Terms

The below terms are discussed to illustrate meanings of the terms as used in this specification, in addition to the understanding of these terms by those of skill in the art. As used herein and in the appended claims, the singular forms “a,” “an,” and, “the” include plural referents unless the context clearly dictates otherwise. It is further noted that the claims can be drafted to exclude any optional element. As such, this statement is intended to serve as antecedent basis for use of such exclusive terminology as “solely,” “only,” and the like in connection with the recitation of claim elements, or use of a “negative” limitation.

As used herein, the term “about” a number refers to that number plus or minus 10% of that number. The term “about” a range refers to that range minus 10% of its lowest value and plus 10% of its greatest value.

As used herein, the terms “subject,” “individual”, and “patient” are used interchangeably. None of the terms are to be interpreted as requiring the supervision of a medical professional (e.g., a doctor, nurse, physician's assistant, orderly, hospice worker). As used herein, the subject may be any animal, including mammals (e.g., a human or non-human animal) and non-mammals. In one embodiment, the subject is a human.

As used herein, the terms “treat,” “treating”, or “treatment,” and other grammatical equivalents, include ameliorating or preventing the underlying causes of one or more symptoms of a disease or condition; alleviating, abating, or ameliorating one or more symptoms of a disease or condition; ameliorating, preventing, or reducing the appearance, severity, or frequency of one or more symptoms of a disease or condition; inhibiting the disease or condition, such as, for example, arresting the development of the disease or condition, relieving the disease or condition, causing regression of the disease or condition, relieving a condition caused by the disease or condition, or inhibiting the symptoms of the disease or condition either prophylactically and/or therapeutically.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer to a material, such as a carrier, or diluent, which does not abrogate the biological activity or properties of the compound, and is relatively nontoxic, e.g., the material may be administered to an individual without causing undesirable biological effects or interacting in a deleterious manner with any of the components of the composition in which it is contained.

“Pharmaceutically acceptable excipient” as used herein, refers to any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives, or lubricants used in formulating pharmaceutical products.

The terms “effective amount” or “therapeutically effective amount,” as used herein, refer to a sufficient amount of an agent or a compound being administered which relieves, to some extent, one or more of the symptoms of the disease or condition being treated, or reduces the underlying cause of the disease or condition being treated. In some aspects, the result is a reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms or underlying cause of the disease (e.g., without undue adverse side effects). In some aspects, an appropriate “effective amount” in any individual case is determined using techniques, such as a dose escalation study. The term “therapeutically effective amount” includes, for example, a prophylactically effective amount. An “effective amount” of a compound disclosed herein may be an amount effective to achieve a desired effect or therapeutic improvement (e.g., without undue adverse side effects). An “effective amount” of a compound disclosed herein may be an amount effective to achieve one or more desired outcomes. It should be understood that, in some cases, “an effective amount” or “a therapeutically effective amount” varies from subject to subject, due to variation in metabolism of the composition, age, weight, general condition of the subject, concomitant medications the subject may be taking, the condition being treated, the severity of the condition being treated, and the judgment of the prescribing physician. In some instances, the disease or condition being treated is familial hypercholesterolemia. In some instances, the disease or condition being treated is homozygous familial hypercholesterolemia. In some instances, the disease or condition being treated is heterozygous familial hypercholesterolemia. In some instances, the disease or condition being treated is a disease or condition associated with or caused by familial hypercholesterolemia.

### Methods of Treating Familial Hypercholesterolemia and Symptoms Thereof

Disclosed herein are methods for treating a subject having, suspected of having, familial hypercholesterolemia or a symptom and/or clinical manifestation thereof (e.g., by alleviating or reducing inflammation and/or oxidative stress induced by (oxidized) LDL (e.g., oxysterol, etc.), and/or by reducing an amount and/or size of circulating cholesterol crystals (and/or clots comprising cholesterol crystals), and/or changing a shape of circulating cholesterol crystals (and/or clots comprising cholesterol crystals), and/or reducing LDL amount or level and/or promoting renal or hepatogenic cholesterol clearance). In some cases, the symptom and/or clinical manifestation thereof is one or more cutaneous manifestations of familial hypercholesterolemia. The one or more cutaneous manifestations of familial hypercholesterolemia include, without limitation, fatty skin deposit (xanthomas) over the hands, elbows, knees, ankles, and/or around the cornea of the eye, cholesterol deposits in the eyelids (Xanthelasma). Clinical presentation of HoFH may include skin and tendon xanthomas, total cholesterol levels between 500 and 1,000 mg/dL, LDL levels of >500 mg/dl, and childhood onset of symptomatic coronary artery disease, as well as aortic valve and proximal aortic root disease, peripheral artery disease (PAD; including claudication, gangrene), and higher incidence of having cardiovascular artery disease (CAD), and consequently myocardial infarction, angina and congestive ischemic heart failure (MI) and/or cerebrovascular disease including TIA and stroke. In HoFH, skin and tendon xanthomas are seen by age 10, while in HeFH they can appear in young adulthood. Tendon xanthomas are present in more than 70% of patients with FH by age 40 to 50.

Clinical presentation of HeFH include patients present with symptoms or signs of cardiovascular disease or adverse cardiovascular disease events at the age of 30-50 years. Many can be identified by an LDL-C greater than the 90th percentile for age and sex when the test was performed for cardiovascular risk screening.

In some cases, treating a subject as described herein alleviates or reduces inflammation and/or oxidative stress induced by oxidized LDL (e.g., oxysterol, etc.). Familial hypercholesterolemia is often associated with defective LDL receptor (LDLR), and/or with mutation in the gene encoding for the LDL receptor, ApoB, ARH/LDLRAP1, STAP1, ApoE or PCSK9. In a subject affected by the LDLR mutated variant (or LDLR Adaptor Proteinl mutated), the amount and/or level of LDL increases in the extracellular space, subjected to oxidation. Such oxidized LDL can be taken up by scavenger receptors (e.g., CD36), resulting in an inflammatory cell uptake (other than LDLR), cellular inflammation by activating NFkB pathway and subsequent expression of proinflammatory genes or proteins via CD36-mediated signaling pathway, deposition of cholesterol crystal leading to activation of NLRP3 inflammasomes, induction of oxidative stress (e.g., C/EBP homologous protein-mediated ER stress), etc. Thus, in some cases, treatment a subject can alleviate and/or reduce such consequences by inducing or facilitating anti-inflammatory signaling cascades via liver X receptor (LXR)-induced ATP binding cassette transporter (ABC)-mediated reverse cholesterol transport (RCT), and/or reduction of intracellular cholesterol crystal deposition. Also, in some instances, in a subject affected by mutated LDL (ApoB), treating a subject can alleviate and/or reduce such consequences by increasing/inducing/facilitating of cholesterol crystal loading and/or transport of LDL.

In some cases, treating a subject as described herein reduces the size (e.g., average size, maximum size) of circulating (e.g., blood, plasma, serum) cholesterol crystals (and/or clots comprising cholesterol crystals) in the subject. In some cases, the size (e.g., average size, maximum size) of circulating (e.g., blood, serum, plasma) cholesterol crystals (and/or clots comprising cholesterol crystals) is reduced by at least about 10% (e.g., at least about 15%, at least about 20%, at least about 25%, at least about 30%, at least about 35%, at least about 40%, at least about 45%, at least about 50%, or greater) relative to the size (e.g., average size, maximum size) of circulating (e.g., blood, serum, plasma) cholesterol crystals (and/or clots comprising cholesterol crystals) prior to treatment with the 2-hydroxypropyl-beta-cyclodextrin. In some cases, treating a subject as described herein reduces an amount (e.g., concentration) of circulating (e.g., blood, plasma, serum) cholesterol crystals (and/or clots comprising cholesterol crystals) in the subject. In some cases, the amount (e.g., concentration) of circulating (e.g., blood, serum, plasma) cholesterol crystals (and/or clots comprising cholesterol crystals) is reduced by at least about 10% (e.g., at least about 15%, at least about 20%, at least about 25%, at least about 30%, at least about 35%, at least about 40%, at least about 45%, at least about 50%, or greater) relative to the amount (e.g., concentration) of circulating (e.g., blood, serum, plasma) cholesterol crystals (and/or clots comprising cholesterol crystals) prior to treatment with the 2-hydroxypropyl-beta-cyclodextrin. In some cases, treating a subject as described herein leads to dissolution of circulating (e.g., blood, plasma, serum) cholesterol crystals (and/or clots comprising cholesterol crystals) in the subject. In some cases, treating a subject as described herein results in a change in shape of circulating cholesterol crystals (and/or clots comprising cholesterol crystals). In some cases, the amount (e.g., concentration) of cholesterol (and/or cholesterol derivates) in urine and/or stool is increased by at least about 10% (e.g., at least about 15%, at least about 20%, at least about 25%, at least about 30%, at least about 35%, at least about 40%, at least about 45%, at least about 50%, at least about 75%, at least about 100%, at least about 200%, or greater, relative to the amount (e.g., concentration) of cholesterol secreted prior to treatment with the 2-hydroxypropyl-beta-cyclodextrin. In some cases, reducing the number and/or size, and/or changing the shape of circulating cholesterol crystals (and/or clots comprising cholesterol crystals) and/or promoting cholesterol clearance in the subject ameliorates or reduces the severity or symptoms of familial hypercholesterolemia in the subject. In some cases, the treatment reduces severity or a symptom of familial hypercholesterolemia in the individual or reduces the clinical manifestation of cardiovascular disease or incidence of major cardio-and cerebrovascular complications including, but not limited to, stroke, TIA, angina pectoris, myocardial infarction, ischemic heart failure, claudication (intermittens) or gangrene. In some cases, reducing the number and/or size, and/or changing the shape of circulating cholesterol crystals (and/or clots comprising cholesterol crystals) in the subject ameliorates or reduces one or more symptoms and/or clinical manifestations of familial hypercholesterolemia in the subject. In some cases, treating a subject as described herein results in a decrease in inflammation (e.g., as measured by, e.g., cytokine protein and/or RNA levels) as compared to a level of inflammation prior to treatment with the 2-hydroxypropyl-beta-cyclodextrin. In some cases, treating a subject as described herein results in an improvement in dermatologic manifestations as compared to prior to treatment with the 2-hydroxypropyl-beta-cyclodextrin.

In some aspects, treating a subject as described herein reduces the need of or dosage of other cholesterol-reducing agents or drugs to the subject. In some instances, the cholesterol-reducing agents or drugs includes, but not limited to, statin (e.g., atorvastatin, Fluvastatin, lovastatin, pitavastatin, pravastatin, rosuvastatin, simvastatin, etc.), cholesterol uptake inhibitors (e.g., Ezetimibe) or PCSK9 inhibitor (PCSK9i, e.g., Evolocumab, Alirocumab, etc.). In some aspects, treating the subject as described herein can eliminate the need of the treatment with statin, cholesterol uptake inhibitor (for instance Ezetimibe) or PCSK9i. In some aspects, treating the subject as described herein can reduce the dosage (e.g., size, amount, frequency, etc.) of the statin or PCSK9i treatment at least 5%, at least 10%, at least 20%, at least 30%, at least 40%, at least 50%, at least 60%, at least 70%, at least 80%, at least 90%. For example, treating the subject as described herein can decrease the amount of statin, cholesterol uptake inhibitors, or PCSK9i per treatment at least 5%, at least 10%, at least 20%, at least 30%, at least 40%, at least 50%, at least 60%, at least 70%, at least 80%, at least 90%. Alternatively and/or additionally, treating the subject as described herein can decrease the frequency of statins, cholesterol uptake inhibitors or PCSK9i per treatment at least 5%, at least 10%, at least 20%, at least 30%, at least 40%, at least 50%, at least 60%, at least 70%, at least 80%, at least 90%, e.g., from every day to every 3 day, from every day to every 5 days, from every day to every 7 days, from every 7 days to every 2 weeks, from every 2 weeks to every 4 weeks, from every 4 weeks to 6 weeks or more, etc. In certain instances, treating the subject as described herein maintains the effect of the treatment with statins, cholesterol uptake inhibitors, or PCSK9i to the subject at least at least 60%, at least 70%, at least 80%, at least 90%, or even increases the effect at least 5%, at least 10%, at least 20%, at least 30%, at least 40%, or at least 50%. Alternatively and/or additionally, treating the subject as described herein can decrease the frequency of or delaying plasmapheresis treatment to the subject diagnosed with or suspected to have familial hypercholesterolemia at least 5%, at least 10%, at least 20%, at least 30%, at least 40%, at least 50%, at least 60%, at least 70%, at least 80%, at least 90%, e.g., from every day to every 3 day, from every day to every 5 days, from every day to every 7 days, from every 7 days to every 2 weeks, from every 2 weeks to every 4 weeks, from every 4 weeks to more than 6 weeks, etc.

In various aspects, the methods involve administering a cyclodextrin to a subject (e.g., having, suspected of having, or at risk of developing familial hypercholesterolemia and/or one or more diseases or conditions associated therewith). Cyclodextrins are a family of cyclic oligosaccharides, consisting of a cyclic (e.g., macrocyclic) ring of glucose subunits joined by α-1,4 glycosidic bonds. Cyclodextrins contain a number of glucose monomers in a ring formation. Common cyclodextrins include alpha-cyclodextrins (consisting of six glucose monomers), beta-cyclodextrins (consisting of seven glucose monomers), gamma-cyclodextrins (consisting of eight glucose monomers), and delta-cyclodextrins (consisting of nine glucose monomers). The outer portion of the ring structure is hydrophilic and the inner cavity of the ring structure is hydrophobic; thus, cyclodextrins generally are water soluble (e.g., due to the hydrophilic exterior), and capable of incorporating hydrophobic molecules in the cavity (e.g., due to the hydrophobic cavity). Parent cyclodextrins have limited water solubility; therefore, several chemically modified cyclodextrins have been synthesized where the hydroxyl groups are substituted with other chemical moieties to, e.g., increase solubility. In various aspects, the methods provided herein involve administering a cyclodextrin to a subject (e.g., a human) in need thereof (e.g., having an elevated amount of circulating cholesterol crystals (and/or clots comprising cholesterol crystals); e.g., having, suspected of having familial hypercholesterolemia). In some cases, the subject has, is suspected of having, or is at risk of developing circulating cholesterol crystals (and/or clots comprising cholesterol crystals) or elevated levels of circulating cholesterol crystals (and/or clots comprising cholesterol crystals) (e.g., after rupture of an atherosclerotic plaque),In some aspects, any atom of the cyclodextrins described herein (e.g., 2-hydroxypropyl-beta-cyclodextrin) may be substituted with any suitable isotope. In a particular embodiment, any one or more hydrogen atoms of the cyclodextrins described herein (e.g., 2-hydroxypropyl-beta-cyclodextrin) may be substituted or replaced with deuterium atoms. Such cyclodextrins are expected to have similar or improved properties as compared to the original cyclodextrin that does not contain deuterium. Deuterium is a safe, stable, non-radioactive isotope of hydrogen. Compared to hydrogen, deuterium forms stronger bonds with carbon. In some instances, the increased bond strength imparted by deuterium can positively impact properties of the cyclodextrins, creating the potential for improved drug efficacy, safety, and/or tolerability. In addition, deuteration may cause decreased metabolic clearance in vivo, thereby increasing the half-life and circulation of the compound. At the same time, because the size and shape of deuterium are essentially identical to those of hydrogen, replacement of hydrogen by deuterium would not be expected to affect the biochemical potency and selectivity of the compound as compared to the original chemical entity that contains only hydrogen.

In particular aspects, the cyclodextrin is 2-hydroxypropyl-beta-cyclodextrin (HPBCD), also known as hydroxypropyl betadex and hydroxypropyl beta-cyclodextrin. In some instances, the 2-hydroxypropyl-beta-cyclodextrin is selected from the group consisting of: Kleptose® HP Parenteral Grade (Roquette Frères, #346114; accessible at roquette.com/-/media/roquette-sharepoint-libraries/sdol_product-specification-sheet/roquette quality specification-sheet kleptose-hp-parenteral-grade_50_346114_en.pdf as of Aug. 26, 2020), Kleptose® HPB Parenteral Grade (Roquette Frères, #346111; accessible at roquette.com/-/media/roquette-sharepoint-libraries/sdol_product-specification-sheet/roquette_quality_specification-sheet_kleptose-hpb-parenteral-grade_50_346111_en.pdf as of Aug. 26, 2020), Kleptose® HPB-LB Parenteral Grade (Roquette Frères, #346115; accessible at roquette.com/-/media/roquette-sharepoint-libraries/sdol_product-specification-sheet/roquette_quality_specification-sheet_kleptose-hpb-lb-parenteral-grade_50_346115_en.pdf as of Aug. 26, 2020), Cavitron® W7 HP5 Pharma cyclodextrin (Ashland; accessible at ashland.com/file_source/Ashland/Product/Documents/Pharmaceutical/PC_11734_Cavitron Cavasol. pdf as of Aug. 26, 2020), Cavitron® W7 HP7 Pharma cyclodextrin (Ashland; accessible at ashland.com/file_source/Ashland/Product/Documents/Pharmaceutical/PC_11734_Cavitron Cavasol. pdf as of Aug. 26, 2020), Trappsol® Cyclo™ (Cyclo Therapeutics, Inc.; accessible at cyclotherapeutics.com/cyclodextrins/trappsol-cyclo as of Aug. 26, 2020), and VTS-270/adrabetadex.

In some aspects, the cyclodextrin molecules or compositions comprising the cyclodextrin molecules described herein (e.g., 2-hydroxypropyl-beta-cyclodextrin) may comprise a β-cyclodextrin cavity. In some aspects, the β-cyclodextrin cavity may comprise a cavity consisting of seven 1.4-linked glucose units. In some embodiments, one or more hydrogen atoms the cyclodextrins described herein (e.g., 2-hydroxypropyl-beta-cyclodextrin) may be substituted with a substituent. In some embodiments, the substituent may be a 2-hydroxypropyl unit. In some aspects, the cyclodextrin molecules described herein may be characterized by average degree of substitution. The term “degree of substitution,” or “DS,” refers to the total number of substituents substituted directly or indirectly on a beta-cyclodextrin molecule. The term “average degree of substitution.” or “average DS” refers to the total number of substituents in a population of beta-cyclodextrin molecules divided by the number of beta-cyclodextrin molecules. In some embodiments, the average DS of the molecule is measured using Electron Spray Ionization-Mass Spectrometry (ESI-MS) analysis (e.g., HPLC-ESI-MS, etc.). In some embodiments, average degree of substitution of the cyclodextrin molecules described herein (e.g., 2-hydroxypropyl-beta-cyclodextrin) is from 1 to 16, from 2 to 14, from 3 to 12, from 4 to 10, from 6 to 8. In some aspects, the average DS of the molecule is determined by peak heights of an electrospray MS spectrum. In some aspects, the average DS of the molecule is determined by multiplying the MS by 7. In some aspects, the cyclodextrin molecules or compositions provided herein contain a plurality of beta-cyclodextrin molecules having an average MS of at least about 0.3. In some aspects, the cyclodextrin molecules or compositions provided herein contain a plurality of beta-cyclodextrin molecules having an average MS of about 0.3 to 1.2. In some aspects, the (e.g., pharmaceutical) compositions provided herein contain a plurality of beta-cyclodextrin molecules having an average MS of 0.8-1.2. In particular aspects, the cyclodextrin molecules described herein (e.g., 2-hydroxypropyl-beta-cyclodextrin) may comprise a specific average degree of substitution of 2-hydropropyl units and controlled impurity levels, thereby improving the safety of the product.

In particular aspects, the cyclodextrin is a polymer. In some aspects, the cyclodextrin polymer is an α-cyclodextrin-based polymer, a β-cyclodextrin-based polymer, or a γ-cyclodextrin-based polymer.

In various aspects, a therapeutically effective amount of cyclodextrin or cyclodextrin derivative (e.g., 2-hydroxypropyl-beta-cyclodextrin) is administered to the subject (e.g., FH patients, HoFH patients, HeFH patients). In some aspects, administration of a therapeutically effective amount of 2-hydroxypropyl-beta-cyclodextrin increases a circulating and/or systemic level of one or more derivative of cholesterol as compared to a baseline in the subject before administering the 2-hydroxypropyl-beta-cyclodextrin. In some aspects, administration of a therapeutically effective amount of 2-hydroxypropyl-beta-cyclodextrin reduces the amount and/or size of the cholesterol crystals in the subject as compared to a baseline or before administering the 2-hydroxypropyl-beta-cyclodextrin. In some aspects, administration of a therapeutically effective amount of 2-hydroxypropyl-beta-cyclodextrin increase expression of ABCA1 gene at least 5%, at least 10%, at least 15%, at least 20%, at least 25%, at least 30%, at least 40%, at least 50%, at least 60%, at least 70%, at least 80%, at least 90%, at least 100%, at least 200% in the subject as compared to a baseline before administering the 2-hydroxypropyl-beta-cyclodextrin. In some aspects, administration of a therapeutically effective amount of 2-hydroxypropyl-beta-cyclodextrin maintains lipid homeostasis in the subject. In some aspects, administration of a therapeutically effective amount of 2-hydroxypropyl-beta-cyclodextrin maintains SREBP2 gene expression differing by within about ±5%, within about ±10%, within about ±15%, within about ±20% in the subject as compared to a baseline before administering the 2-hydroxypropyl-beta-cyclodextrin. In some aspects, administration of a therapeutically effective amount of 2-hydroxypropyl-beta-cyclodextrin inhibits increase of liver weight of the subject by less than about 30%, about 25%, about 20%, about 15%, about 10%, or about 5% in the subject as compared to a baseline before administering the 2-hydroxypropyl-beta-cyclodextrin. In some aspects, administration of a therapeutically effective amount of 2-hydroxypropyl-beta-cyclodextrin maintains liver weight of the subject differing by within about ±5%, within about ±10%, within about ±15%, within about ±20% in the subject as compared to a baseline before administering the 2-hydroxypropyl-beta-cyclodextrin. In some aspects, administration of a therapeutically effective amount of 2-hydroxypropyl-beta-cyclodextrin reduces liver weight by at least about 0.5%, at least about 1%, at least about 5%, at least about 10%, at least about 15%, at least about 20%, at least about 25%, at least about 30%, at least about 35%, at least about 40% in the subject as compared to a baseline before administering the 2-hydroxypropyl-beta-cyclodextrin. In some aspects, the one or more derivative of cholesterol is a by-product of cholesterol biosynthesis. In some aspects, the one or more derivative of cholesterol comprises a hydrogenated product, products with differently hydrogenated 1H-cyclopenta[a]phenanthren-3-ol products, or products formed with a hydroxyl, epoxyl, or keto group. In some cases, the one or more derivative of cholesterol is an oxysterol or a sterol.

In some aspects, the therapeutically effective amount of cyclodextrin or cyclodextrin derivative (e.g., 2-hydroxypropyl-beta-cyclodextrin) is an amount suitable to achieve the therapeutic effect described herein. In some aspects, the therapeutically effective amount is at least about 50 mg/kg, at least about 100 mg/kg, at least about 200 mg/kg, at least about 300 mg/kg, at least about 400 mg/kg, at least about 500 mg/kg, at least about 600 mg/kg, at least about 700 mg/kg, at least about 800 mg/kg, at least about 900 mg/kg, at least about 1000 mg/kg, at least about 1100 mg/kg, at least about 1200 mg/kg, at least about 1300 mg/kg, at least about 1400 mg/kg, at least about 1500 mg/kg, at least about 1600 mg/kg, at least about 1700 mg/kg, at least about 1800 mg/kg, at least about 1900 mg/kg, at least about 2000 mg/kg, at least about 2100 mg/kg, at least about 2200 mg/kg, at least about 2300 mg/kg, at least about 2400 mg/kg, at least about 2500 mg/kg, at least about 3500 mg/kg, at least about 3500 mg/kg, at least about 4000 mg/kg, at least about 4500 mg/kg, at least about 5000 mg/kg, at least about 5500 mg/kg, at least about 6000 mg/kg, at least about 6500 mg/kg, at least about 7000 mg/kg, at least about 7500 mg/kg, or at least about 8000 mg/kg. In some aspects, the therapeutically effective amount of cyclodextrin or cyclodextrin derivative (e.g., 2-hydroxypropyl-beta-cyclodextrin) is at least about 100 mg/kg. In some aspects, the therapeutically effective amount of cyclodextrin or cyclodextrin derivative (e.g., 2-hydroxypropyl-beta-cyclodextrin) is at least about 250 mg/kg. In some aspects, the therapeutically effective amount of cyclodextrin or cyclodextrin derivative (e.g., 2-hydroxypropyl-beta-cyclodextrin) is at least about 500 mg/kg. In some aspects, the therapeutically effective amount of cyclodextrin or cyclodextrin derivative (e.g., 2-hydroxypropyl-beta-cyclodextrin) is at least about 1000 mg/kg. In some aspects, the therapeutically effective amount of cyclodextrin or cyclodextrin derivative (e.g., 2-hydroxypropyl-beta-cyclodextrin) is at least about 1500 mg/kg.

In some aspects, the therapeutically effective amount of cyclodextrin or cyclodextrin derivative (e.g., 2-hydroxypropyl-beta-cyclodextrin) is an amount suitable to achieve the therapeutic effect described herein. In some aspects, the therapeutically effective amount is from about 50 mg/kg to about 2500 mg/kg (e.g., from about 50 mg/kg to about 1000 mg/kg, from about 500 mg/kg to about 1000 mg/kg, from about 500 mg/kg to about 1500 mg/kg, from about 800 mg/kg to about 1500 mg/kg, from about 800 mg/kg to about 1200 mg/kg, from about 1000 mg/kg to about 1500 mg/kg, from about 1000 mg/kg to about 2500 mg/kg. In some aspects, the therapeutically effective amount of 2-hydroxypropyl-beta-cyclodextrin is from about 500 mg/kg to about 1500 mg/kg. In some aspects, the therapeutically effective amount of cyclodextrin or cyclodextrin derivative (e.g., 2-hydroxypropyl-beta-cyclodextrin) is from about 800 mg/kg to about 1200 mg/kg.

In some aspects, the therapeutically effective amount of cyclodextrin or cyclodextrin derivative (e.g., 2-hydroxypropyl-beta-cyclodextrin) is an amount suitable for achieving the therapeutic effect described herein. In some aspects, the therapeutically effective amount is at least about 4 g (e.g., at least about 10 g, at least about 25 g, at least about 50 g, at least about 75 g, at least about 100 g, at least about 125 g, at least about 150 g, at least about 175 g, at least about 200 g, at least about 250 g). In some aspects, the therapeutically effective amount of 2-hydroxypropyl-beta-cyclodextrin may be from about 4 g to about 250 g (e.g., from about 4 g to about 200 g, from about 4 g to about 150 g, from about 4 g to about 100 g, from about 4 g to about 50 g, from about 50 g to about 250 g, from about 50 g to about 200 g, from about 50 g to about 150 g, from about 50 g to about 100 g, from about 100 g to about 250 g, from about 100 g to about 200 g). The total amount of cyclodextrin or cyclodextrin derivative (e.g., 2-hydroxpropyl-beta-cyclodextrin) administered (e.g., in a single dose administration, e.g., in a therapeutically effective amount) may depend on a number of factors, including, without limitation, the subject's age, gender, weight, and the like.

In some aspects, the therapeutically effective amount of cyclodextrin or cyclodextrin derivative (e.g., 2-hydroxypropyl-beta-cyclodextrin) is an amount sufficient to achieve a whole blood, serum, and/or plasma concentration of cyclodextrin or cyclodextrin derivative (e.g., 2-hydroxypropyl-beta-cyclodextrin) suitable for achieving the therapeutic effect described herein. In some aspects, the whole blood, serum, and/or plasma concentration is at least about 0.01 mM (e.g., at least about 0.05 mM, at least about 0.1 mM, at least about 0.2 mM, at least about 0.3 mM, at least about 0.4 mM, at least about 0.5 mM, at least about 0.6 mM, at least about 0.7 mM, at least about 0.8 mM, at least about 0.9 mM, at least about 1.0 mM, at least about 1.5 mM, at least about 2.0 mM, or at least about 2.5 mM).

The methods disclosed herein may further comprise administering, at a first time point, a therapeutically effective first amount of cyclodextrin or cyclodextrin derivative (e.g., 2-hydroxypropyl-beta-cyclodextrin) to a subject, and administering, at a second time point, a therapeutically effective second amount of cyclodextrin or cyclodextrin derivative (e.g., 2-hydroxypropyl-beta-cyclodextrin) to the subject. The second time point can be at least 4 hours, at least 6 hours, at least 8 hours, at least 12 hours, at least 1 day, at least 2 days, at least 3 days, at least 4 days, at least 5 days, at least 6 days, at least 1 week, at least 2 weeks, at least 3 weeks, or at least 4 weeks after the first time point. In some aspects, the administering further comprises administering every 3 days, every 7 days, every 10 days, every 14 days, every 21 days, every 28 days, every 2 months, every 3 months, every 6 months, every 12 months. In some aspects, the administering continues at least 1 year, at least 2 years, at least 3 years, at least 5 years, at least 7 years, at least 10 years, at least 15 years, at least 20 years, or as long as the cyclodextrin or cyclodextrin derivative (e.g., 2-hydroxypropyl-beta-cyclodextrin) is effective in ameliorating at least one symptom of the familial hypercholesterolemia. In some aspects, the administering may be by intravenous administration.

In some cases, the treatment of cyclodextrin or cyclodextrin derivative (e.g., -hydroxypropyl-beta-cyclodextrin) is combined with other cholesterol-reducing treatment and/or LDL inhibitors (e.g., statins, cholesterol uptake inhibitors, or PSCK9 inhibitor). In some aspects, the cyclodextrin or cyclodextrin derivative with other cholesterol-reducing treatment is co-administered concurrently to the individual. In some aspects, the cyclodextrin or cyclodextrin derivative with other cholesterol-reducing treatment is sequentially administered to the individual.

In some cases, the second time point may be determined based on one or more indicators that an additional dose of drug would be beneficial to the subject. For example, the second time point may be administered after the therapeutic benefit of the first dose has diminished or has started to diminish.

In various aspects, the subject can be a human. In some cases, the subject may be of any age that is at risk of or more prone to developing elevated levels of circulating cholesterol crystals (and/or clots comprising cholesterol crystals) and/or familial hypercholesterolemia. The subject may be under 1, under 3, under 5 years old, or at least 5 years old (e.g., at least 10, at least 15, at least 20, at least 25, at least 30, at least 40). The subject can be diagnosed with atherosclerosis and/or atherosclerotic cardiovascular disease. In some cases, the subject has advanced atherosclerosis. In some cases, the subject has undergone a medical procedure involving the blood vessels, such as vascular surgery or angiography. In some cases, the subject has commenced treatment with an anticoagulant or a thrombolytic medication. The subject may have one or more risk factors for 5 (e.g., at least 10, at least 15, at least 20, at least 25, at least 30, at least 40). Risk factors for familial hypercholesterolemia include, but are not limited to, a family history of familial hypercholesterolemia, high level of LDL cholesterol in at least one parent, a change in LDLR gene, ApoB gene, or PCSK9 gene.

The subject can be diagnosed with familial hypercholesterolemia and/or may have a symptom and/or a clinical manifestation of familial hypercholesterolemia, or has manifestations in consanguineous family. Familial hypercholesterolemia can be diagnosed by a genetic screening test or in combination with other clinical tests e.g., a biopsy (e.g., a skin biopsy, a muscle biopsy, a kidney biopsy, bone marrow biopsy, gastric mucosa biopsy, colonic mucosa biopsy) or typical dermatological manifestations including xanthoma, and xanthelasma or other signs of poor skin perfusion at an early age (including gangrene, livedo reticularis, cyanotic toes or fingers), and or histological manifested accumulation of foamy macrophages in skin or plaque lesions, increased total cholesterol levels, increased LDL levels, with atherosclerotic disease comorbidity or incidences thereof, such as atherosclerotic peripheral artery, coronary artery and/or cerebrovascular disease including angina pectoris, myocardial infarction, claudication, TIA and/or stroke. In some cases, the subject can be diagnosed by a combination of characteristic manifestations of the disease including significant LDL-C levels or early onset atherosclerotic disease with a strong familial predisposition, or dermatological signs (e.g., including xanthomas, xanthelasma, cutaneous, renal, central nervous system, ocular manifestations (e.g., Hollenhorst plaques), e.g., as described herein). In some cases, the subject can be diagnosed by a non-invasive imaging modality (e.g., abdominal ultrasound, chest/abdominal computerized tomography (CT), transthoracic echocardiogram (TTE), transesophageal echocardiogram (TEE) showing early-onset or rapid progressive atherosclerosis).

The subject can have one or more analytical laboratory results consistent with familial hypercholesterolemia. The one or more analytical laboratory results consistent with familial hypercholesterolemia may include, without limitation, high total cholesterol (up to 200 mg/dL, up to 250 mg/dL, up to 300 mg/dL, up to 350 mg/dL, up to 400 mg/dL, up to 500 mg/dl, up to 600 mg/dl, up to 700 mg/dl, up to 800 mg/dl, up to 900 mg/dl, up to 1,000 mg/dl), high LDL levels (>200 mg/dL, >250 mg/dL, >300 mg/dL, >350 mg/dL, >400 mg/dL, >500 mg/dl). In some cases, familial hypercholesterolemia involves secondary manifestations. In some aspects, LDL >200mg/dl, >300 mg/dl, >400 mg/dl, or >500 mg/dl, and/or and total cholesterol >650-1000 mg/d1 or premature coronary heart disease (<55 years men; <60 years women) in a first degree relative is sufficient for a clinical diagnosis of FH. DNA tests can confirm the diagnosis in 80% of cases. Since cholesterol levels are elevated from early childhood, in some aspects, children are screened before the age of 1, 2, 3, 4, 5, age 10, age 20, age 30, etc.

The subject may be treated (e.g., by the methods described herein) before developing symptoms of familial hypercholesterolemia. For example, a subject at risk of developing familial hypercholesterolemia (e.g., a subject with elevated levels of circulating cholesterol crystals (and/or clots comprising cholesterol crystals), a family history of familial hypercholesterolemia, high level of LDL cholesterol in at least one of parents, a change in LDLR gene or LDLR adapter protein 1 (LDLRAP1), ApoB gene, or PCSK9 gene, may be treated (e.g., by the methods described herein), e.g., to decrease the amount of cholesterol and/or LDL, and/or size of, and/or change the shape of the circulating cholesterol crystals (and/or clots comprising cholesterol crystals) (e.g., thereby reducing the severity or symptoms of familial hypercholesterolemia). In some cases, a subject having one or more risk factors for familial hypercholesterolemia is treated prior to developing elevated levels of circulating cholesterol to prevent further development of atherosclerotic coronary artery, peripheral artery or cerebrovascular disease. The subject may be treated (e.g., by the methods described herein) after developing familial hypercholesterolemia and/or a symptom or clinical manifestation thereof

The methods disclosed herein can be used to treat familial hypercholesterolemia and/or one or more symptoms and/or clinical manifestations thereof. For example, the methods disclosed herein can be used to treat progressive coronary artery disease (and ischemic cardiomyopathy), ischemic cerebrovascular disease, or cutaneous manifestations of familial hypercholesterolemia (e.g., xanthomas, xanthelasma, livedo reticularis, cyanosis).

In some aspects, the methods described herein cause, or lead to a reduction in the size (e.g., average size, maximum size) of circulating (e.g., blood, plasma, serum) cholesterol crystals (and/or clots comprising cholesterol crystals) in the subject. In some cases, the size (e.g., average size, maximum size) of circulating (e.g., blood, plasma, serum) cholesterol crystals (and/or clots comprising cholesterol crystals) may be reduced relative to the size (e.g., average size, maximum size) of the circulating (e.g., blood, plasma, serum) cholesterol crystals (and/or clots comprising cholesterol crystals) prior to the treating. In some aspects, the size (e.g., average size, maximum size) of circulating (e.g., blood, plasma, serum) cholesterol crystals (and/or clots comprising cholesterol crystals) may be reduced by at least about 0.5%. In some aspects, the size (e.g., average size, maximum size) of circulating (e.g., blood, plasma, serum) cholesterol crystals (and/or clots comprising cholesterol crystals) may be reduced by at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or greater.

In some aspects, the methods described herein causes, or lead to an increase of ABCA1 gene expression in the subject. In some cases, ABCA1 gene expression in the subject is increased by at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or greater. In some cases, the increase of ABCA1 gene expression is not accompanied with increase or decrease of SREBP2 gene expression.

In some aspects, the methods described herein causes, or lead to reduction of liver weight by at least about 0.5%, at least about 1%, at least about 5%, at least about 10%, at least about 15%, at least about 20%, at least about 25%, at least about 30%, at least about 35%, at least about 40%.

In some aspects, the methods described herein causes, or lead to a reduction in the amount (e.g., concentration) of circulating (e.g., blood, plasma, serum) cholesterol crystals (and/or clots comprising cholesterol crystals) in the subject. In some cases, the circulating cholesterol crystals derive from abundant LDL levels resulting in uncontrolled intra- or extracellular cholesterol crystal deposition. In some cases, the amount (e.g., concentration) of circulating (e.g., blood, plasma, serum) cholesterol crystals (and/or clots comprising cholesterol crystals) may be reduced relative to the amount (e.g., concentration) of circulating (e.g., blood, plasma, serum) cholesterol crystals (and/or clots comprising cholesterol crystals) prior to the treating. In some aspects, the amount (e.g., concentration) of circulating (e.g., blood, plasma, serum) cholesterol crystals (and/or clots comprising cholesterol crystals) may be reduced by at least about 0.5%. In some aspects, the amount (e.g., concentration) of circulating (e.g., blood, plasma, serum) cholesterol crystals (and/or clots comprising cholesterol crystals) may be reduced by at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or greater. In some aspects, the methods described herein result in a change in the shape of circulating cholesterol crystals (and/or clots comprising cholesterol crystals).

In some cases, the methods described herein cause, or lead to a decrease in inflammation (e.g., as measured by, e.g., cytokine protein and/or RNA levels) as compared to a level of inflammation prior to treatment with the 2-hydroxypropyl-beta-cyclodextrin. In some cases, the methods described herein result in an improvement in dermatologic manifestations as compared to prior to treatment with the 2-hydroxypropyl-beta-cyclodextrin.

In some aspects, the methods involve treating a subject (e.g., having, suspected of having, or at risk of developing familial hypercholesterolemia) with a combination of 2-hydroxypropyl-beta-cyclodextrin and an additional treatment (e.g., therapeutic pharmaceutical compound, medical procedure, or therapy).

In some cases, the additional treatment comprises pheresis (e.g., plasmapheresis). In some cases, the additional treatment comprises cholesterol lowering treatment. In some cases, the additional treatment comprises LDL-lowering treatment or LDL-lowering medicaments. In some cases, the additional treatment comprises therapeutics that is selected from the group consisting of: a HMG-CoA reductase inhibitor (statin), cholesterol uptake inhibitor (for instance ezetimibe, fibrin), lomitapide, an anti-inflammatory drug (e.g., low-dose acetyl salicylic acid (ASS), canakinumab), a corticosteroid, and a proprotein convertase subtilisin/kexin type 9 (PCSK9) inhibitor and/or plasmapheresis. In some cases, the additional treatment comprises two or more treatment, medicaments, medical procedures described above.

In some cases, 2-hyroxypropyl-beta-cyclodextrin and the additional therapeutic are administered to the subject at or near the same time (e.g., in a single formulation, or as separate formulations). In some cases, 2-hydroxypropyl-beta-cyclodextrin and the additional therapeutic are administered at different times (e.g., in separate formulations). In some cases, the additional therapeutic is administered prior to administration with 2-hydroxypropyl-beta-cyclodextrin. In some cases, the additional therapeutic is administered concurrently with 2-hydroxypropyl-beta-cyclodextrin. In some cases, the additional therapeutic is administered after administration of 2-hydroxypropyl-beta-cyclodextrin.

In some cases, the subject may have previously been undergoing treatment with an additional therapeutic (e.g., prior to administration with 2-hydroxypropyl-beta-cyclodextrin). In some cases, the treatment with the additional therapeutic may be ineffective or may have limited efficacy. In such cases, subjects treated with 2-hydroxypropyl-beta-cyclodextrin (e.g., after treatment with the additional therapeutic, or concurrently with the additional therapeutic) may exhibit a greater therapeutic benefit than administration of the additional therapeutic alone.

In some cases, subjects treated with both 2-hydroxypropyl-beta-cyclodextrin and an additional therapeutic may exhibit a therapeutic benefit greater than the therapeutic benefit exhibited by treatment with either the additional therapeutic or the 2-hydroxypropyl-beta-cyclodextrin alone. In some cases, treatment with both the additional therapeutic and 2-hydroxypropyl-beta-cyclodextrin has a synergistic effect, such that the interaction between the additional therapeutic and 2-hydroxypropyl-beta-cyclodextrin causes the total effect of the therapeutics to be greater than the sum of the individual effects of each therapeutic. In some cases, treatment with both the additional therapeutic and 2-hydroxypropyl-beta-cyclodextrin has an additive effect.

### Pharmaceutical Compositions

Disclosed herein, in certain aspects, are pharmaceutical compositions comprising an amount of 2-hydroxypropyl-beta-cyclodextrin effective to treat familial hypercholesterolemia and/or one or more symptoms and/or clinical manifestations thereof, in a human; and an excipient. The excipient can be a pharmaceutically acceptable excipient.

The excipient may comprise a tonicity adjusting agent, a preservative, a solubilizing agent, a buffer, a solution (e.g., an IV solution), or any combination thereof. The tonicity adjusting agent can be dextrose, glycerol, sodium chloride, glycerin, mannitol, or a combination thereof. The preservative can be an antioxidant, an antimicrobial, a chelating agent, or a combination thereof. The antioxidant can be ascorbic acid, acetylcysteine, a sulfurous acid salt (e.g., bisulfite, metabisulfite), a monothioglycerol, or a combination thereof. The antimicrobial can be a phenol, meta-cresol, benzyl alcohol, paraben, benzalkonium chloride, chlorobutanol, thimerosal, phenylmercuric salts (e.g., acetate, borate, nitrate), or a combination thereof. The chelating agent can be calcium disodium ethylenediaminetetraacetic acid (EDTA), disodium EDTA, sodium EDTA, calcium versetamide sodium, calteridol, diethylenetriaminepenta acetic acid (DTPA), or a combination thereof. The solubilizing agent can be a surfactant or a co-solvent. The surfactant can be polyoxyethylene sorbitan monooleate (Tween 80), sorbitan monooleate polyoxyethylene sorbitan monolaurate (Polysorbat 20, Tween® 20), lecithin, polyoxyethylene-polyoxypropylene copolymers (Pluronics), or a combination thereof. The co-solvent can be propylene glycol, glycerin, ethanol, polyethylene glycol (PEG), sorbitol, dimethylacetamide, Cremophor EL, or a combination there. The polyethylene glycol can be PEG 300, PEG 400, PEG 600, PEG 3350, or PEG 4000. The buffer can comprise sodium acetate, acetic acid, glacial acetic acid, ammonium acetate, ammonium sulfate, ammonium hydroxide, arginine, aspartic acid, benzene sulfonic acid, benzoate sodium, benzoic acid, sodium bicarbonate, boric acid, sodium boric acid, sodium carbonate, citrate acid, sodium citrate, disodium citrate, trisodium citrate, diethanolamine, glucono delta lactone, glycine, glycine HCl, histidine, histidine HCl, hydrochloric acid, hydrobromic acid, lysine, maleic acid, meglumine, methanesulfonic acid, monoethanolamine, phosphate acid, monobasic potassium, dibasic potassium, monosodium phosphate, disodium phosphate, trisodium phosphate, sodium hydroxide, succinate sodium, sulfuric acid, tartarate sodium, tartaric acid, tromethamine (Tris), or a combination thereof.

The pharmaceutical composition can comprise at least about 4 g, at least about 10 g, at least about 50 g, at least about 100 g, at least about 150 g, at least about 200 g, or at least about 250 g of 2-hydroxypropyl-beta-cyclodextrin. In some aspects, the pharmaceutical composition comprises at least about 4 g of 2-hydroxypropyl-beta-cyclodextrin. In some aspects, the pharmaceutical composition comprises at least about 50 g of 2-hydroxypropyl-beta-cyclodextrin. In some aspects, the pharmaceutical composition comprises at least about 100 g of 2-hydroxypropyl-beta-cyclodextrin. In some aspects, the pharmaceutical composition comprises at least about 200 g of 2-hydroxypropyl-beta-cyclodextrin. In some aspects, the pharmaceutical composition comprises from about 4 g to about 250 g of 2-hydroxypropyl-beta-cyclodextrin (e.g., from about 4 g to about 100 g, from about 4 g to about 50 g, from about 50 g to about 150 g, from about 50 g to about 250 g, from about 100 g to about 200 g, from about 100 g to about 250 g, from about 150 g to about 250 g).

The pharmaceutical composition can be formulated for single dose administration. The pharmaceutical composition can be formulated for intravenous administration. The pharmaceutical composition can be formulated to be isotonic.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

### EXAMPLES

### Example 1

### Cholesterol Crystal Dissolution Capacity in HoFH Patients

D6-labeled cholesterol crystals were added to human plasma collected from homozygous familial hypercholesterolemia (HoFH) patients treated with HPB CD (2-hydroxypropyl-beta-cyclodextrin). The reaction mixtures were filtered to remove crystals and the filtrate was assayed for the presence of D6-labeled cholesterol using gas chromatography-mass spectroscopy (GC-MS). Cholesterol measured in the filtrate indicated the ability of HPBCD (2-hydroxypropyl-beta-cyclodextrin) to dissolve cholesterol crystals.

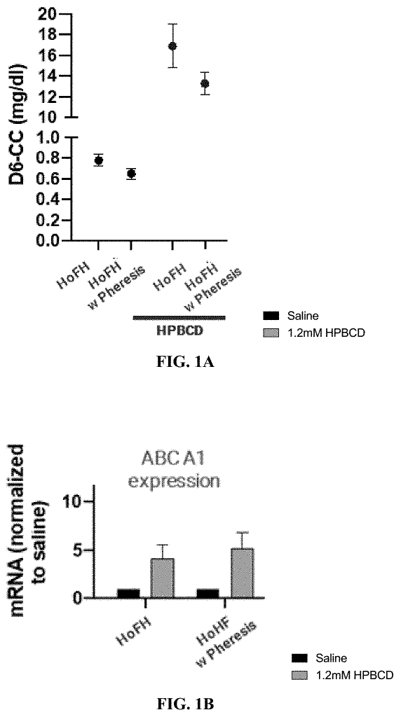

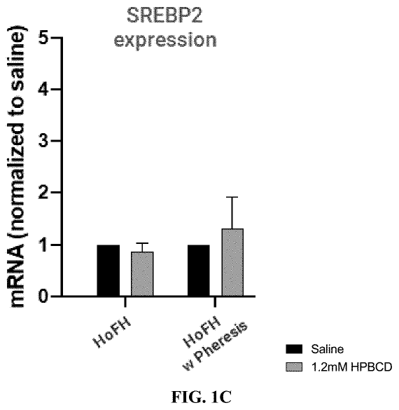

As shown in FIG. 1A, HPBCD (2-hydroxypropyl-beta-cyclodextrin) significantly increased cholesterol crystal dissolution compared to control group. This example indicates that HPBCD (2-hydroxypropyl-beta-cyclodextrin) has therapeutic potential to remove cholesterol crystals from the circulation. The dissolution of cholesterol crystals in HoFH patients will reduce inflammation in atheromas. By mobilizing cholesterol from the circulation and from tissue, plaque progression can be reduced and plaque stability can be improved. This would significantly reduce the incidence of significant atheroma-related adverse clinical outcomes such as cardiovascular death, myocardial infarction, or hospitalization for heart failure.

### Example 2

### Effects on Cholesterol Efflux Capacity in HoFH Patients

This example aims to investigate the effects of HPBCD (2-hydroxypropyl-beta-cyclodextrin) on cholesterol efflux capacity in HoFH patients. HPBCD was added to whole blood obtained from HoFH patients at concentration of 1.2 mM. Untreated samples were used as control. The mixtures were incubated for 6 hours. Total mRNA was isolated and mRNA expression levels were measured. Data is shown as the level of gene expression normalized to saline control.

As shown in FIG. 1B, HPBCD (2-hydroxypropyl-beta-cyclodextrin) caused a significant induction in the reverse cholesterol transporter ABCA1 in human whole blood. LXR targeting is generally associated with upregulation of SREBP2. However, as shown in FIG. 1C, mRNA expression level of SREBP2 in HoFH patients treated with HPBCD is similar to control group. This example illustrates that HPBCD (2-hydroxypropyl-beta-cyclodextrin can cause the removal of excess cholesterol from cells without increasing circulating cholesterol levels (i.e., maintaining cholesterol homeostasis).

### Example 3

### Effect on Liver Weight in Mouse Model

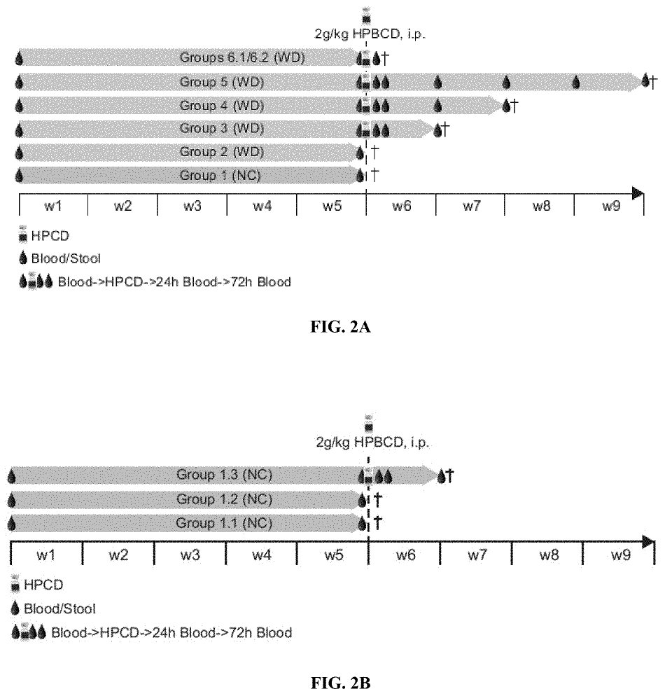

To evaluate the effect of HPBCD (2-hydroxypropyl-beta-cyclodextrin) on liver weight, experiments using Ldlr−/− mice were designed as described in FIGS. 2A and 2B. The mice were fed normal chow diet (NC) or high-fat (42%), high-cholesterol (1.2) diet Western diet (WD) as described in Table 1 and Table 2. After five weeks, the mice were subcutaneously injected with 2 g/kg BPBCD. The livers were excised and weighed after five weeks (Group 1, 2, 6), six weeks (Group 3), seven weeks (Group 4), and nine weeks (Group 5), respectively.

TABLE 1

| | | | | | Group |
|---|---|---|---|---|---|
| Group 1 | Group 2 | Group 3 | Group 4 | Group 5 | 6.1/6.2 |
| 5 weeks | 5 weeks | 6 weeks | 7 weeks | 9 weeks | 5 weeks WD |
| Chow diet | WD | WD | WD | WD | Control (6.2) |
| | | 1 week | 2 weeks | 4 weeks | 24 post- |
| | | post- | post- | post- | HPBCD (6.1) |
| | | HPBCD | HPBCD | HPBCD | |

TABLE 2

| Group 1.1 | Group 1.2 | Group 1.3 |
|---|---|---|
| 5 weeks Chow diet | 5 weeks Chow diet | 5 weeks Chow diet |
| 24h post HPBCD | | 1 week post-HPBCD |

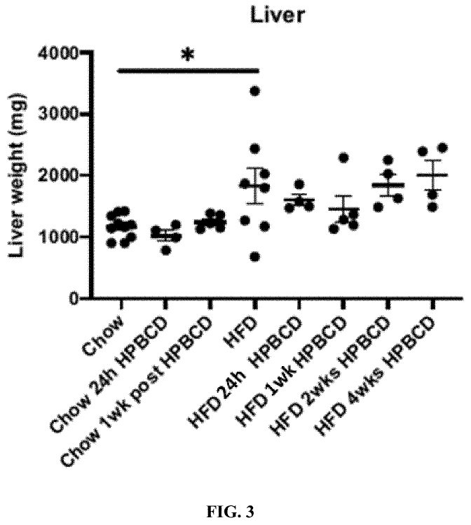

As shown in FIG. 3, the untreated mice group showed a significant increase in liver weight, but HPBCD-treated mice did not show any further increase in liver weight by high fat diet. This example suggests that HPBCD (2-hydroxypropyl-beta-cyclodextrin) could effectively inhibit increase of liver weight in FH patients.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of i) alleviating or reducing inflammation and/or oxidative stress induced by oxidized LDL, ii) reducing an amount total cholesterol, iii) reducing accumulation of total LDL, iv) reducing an amount of and/or a size of, and/or changing the shape of circulating cholesterol crystals, and/or v) improving the renal and/or hepatogenic clearance of cholesterol in an individual, the method comprising: administering a therapeutically effective amount of cyclodextrin or a derivative thereof to the individual, thereby alleviating or reducing inflammation and/or oxidative stress induced by oxidized LDL, and/or reducing the amount of and/or size of, and/or changing the shape of circulating cholesterol crystals, and/or promoting renal hepatogenic clearance of cholesterol derivates in the individual diagnosed with or suspected to have familial hypercholesterolemia.

2. The method of claim 1, wherein the size of circulating cholesterol crystals is reduced by at least about 10% relative to the size of circulating cholesterol crystals prior to treatment with the cyclodextrin or derivative thereof

3. The method of claim 1, wherein promoting renal hepatogenic clearance of cholesterol derivates comprises increasing renal or hepatogenic clearance of cholesterol or cholesterol derivates in the individual by at least about 10% relative to the amount of cholesterol and/or cholesterol derivates cleared prior to the treatment.

4. The method of claim 1, wherein the amount of circulating cholesterol crystals is reduced by at least about 10% relative to the amount of circulating cholesterol crystals prior to treatment with the cyclodextrin or the derivative thereof.

5. (canceled)

6. The method of claim 1, wherein the cyclodextrin or derivative thereof comprises 2-hydroxypropyl-beta-cyclodextrin.

7. A method of treating familial hypercholesterolemia or one or more symptoms thereof in an individual, or reducing a complication related to familial hypercholesterolemia, the method comprising: administering a therapeutically effective amount of cyclodextrin or derivative thereof to the individual, thereby treating the familial hypercholesterolemia and/or one or more symptoms thereof in the individual.

8. (canceled)

9. The method of claim 7, wherein the cyclodextrin or derivative thereof comprises 2-hydroxypropyl-beta-cyclodextrin, Kleptose® HP Parenteral Grade, Kleptose® HPB Parenteral Grade, Kleptose® HPB-LB Parenteral Grade, Cavitron® W7 HP5 Pharma cyclodextrin, Cavitron® W7 HP7 Pharma cyclodextrin, Trappsol® Cyclo™, and/or VT S-270/adrabetadex.

10. The method of claim 7, wherein the therapeutically effective amount is from about 50 mg/kg to about 8,000 mg/kg, or from about 4 g to about 250 g.

11. (canceled)

12. The method of claim 7, wherein the therapeutically effective amount is an amount sufficient to achieve a serum, plasma, and/or whole blood concentration of 2-hydroxypropyl-beta-cyclodextrin of about 0.01 mM to about 5 mM.

13. (canceled)

14. The method of claim 7, wherein the subject has one or more risk factors for familial hypercholesterolemia, or one or more analytical lab results associated with familial hypercholesterolemia.

15. The method of claim 14, wherein the one or more risk factors for familial hypercholesterolemia is selected from the group consisting of: a family history of familial hypercholesterolemia, high level of LDL cholesterol in at least one of parents or in the subject, a change in LDLR gene, LDLRAP1 gene, ApoB gene, ApoE gene, LDLRAP1/ARH gene, STAP1 gene, or PCSK9 gene.

16. (canceled)

17. The method of claim 14, wherein the one or more analytical lab results associated with familial hypercholesterolemia comprises increased serum/plasma total cholesterol, or LDL.

18. (canceled)

19. (canceled)

20. The method of claim 7, wherein the administering further comprises: (i) administering, at a first time point, a therapeutically effective first dose of 2-hydroxypropyl-beta-cyclodextrin to the individual; and (ii) administering, at a second time point, a therapeutically effective second dose of 2-hydroxypropyl-beta-cyclodextrin to the individual.

21. The method of claim 20, wherein the second time point is at least 4 hours, at least 6 hours, at least 8 hours, at least 12 hours, at least 1 day, at least 2 days, at least 3 days, at least 4 days, at least 5 days, at least 6 days, at least 1 week, at least 2 weeks, at least 3 weeks, or at least 4 weeks, 2 months, 3 months, 6 months, 12 months after the first time point.

22. The method of claim 7, wherein the administering further comprises administering every 3 days, every 7 days, every 10 days, every 14 days, every 21 days, every 28 days, every 2 months, every 3 months, every 6 months, every 12 months.

23. The method of claim 7, wherein the administering is by parenteral methods or in conjunction to plasmapheresis.

24.-34. (canceled)

35. A method of i) reducing statins, cholesterol uptake inhibitors, or PCSK9 inhibitor treatment, or ii) reducing a frequency of or delaying plasmapheresis treatment to a subject diagnosed with or suspected to have familial hypercholesterolemia, the method comprising: administering a therapeutically effective amount of cyclodextrin or derivative thereof to the subject, thereby treating the familial hypercholesterolemia and/or one or more symptoms thereof in the subject.

36. The method of claim 35, wherein the cyclodextrin or derivative thereof comprises 2-hydroxypropyl-beta-cyclodextrin.

37.-42. (canceled)

43. The method of claim 35, wherein the statin, cholesterol uptake inhibitor, or PCSK9 inhibitor treatment is reduced at least 30% compared to before administering the cyclodextrin or derivative thereof.

44. The method of claim 35, wherein the frequency of the plasmapheresis treatment is reduced at least 30% compared to before administering the cyclodextrin or derivative thereof, or wherein the plasmapheresis treatment is delayed at least 6 months.

45.-50. (canceled)
